(19) United States
(12) Patent Application Publication
(10) Pub. No.: US 2016/0008070 A1
(43) Pub. Date: Jan. 14, 2016

(54) EXPANDABLE AND FOLDABLE MAYO STAND

(71) Applicant: CONCEPTO LLC, Lebanon, OH (US)

(72) Inventors: Sambhu N. CHOUDHURY, Cincinatti, OH (US); Arturo D. SANCHEZ, Lebanon, OH (US); Sean N. LYNCH, Cincinatti, OH (US)

(21) Appl. No.: 14/678,531

(22) Filed: Apr. 3, 2015

Related U.S. Application Data
(60) Provisional application No. 61/995,110, filed on Apr. 4, 2014.

(51) Int. Cl. A61B 19/02; A47B 3/00; A47B 13/02; A47B 9/20; A47B 13/08

(52) U.S. Cl. CPC A61B 19/0248; A47B 9/20; A47B 13/081; A47B 13/023; A47B 3/002; A61B 2019/0251; A47B 2013/025

(57) ABSTRACT

An expandable and foldable mayo stand has a support column having first and second ends and a longitudinal axis. A table top is connected to the first end of the support column, and a base is connected to the second end of the support column. The table top is formed in two halves having a hinge connection extending along a hinge axis perpendicular to the longitudinal axis, whereby the table top halves can be pivoted between an open position in which they are coplanar and a collapsed position in which they are substantially parallel. The base has a central portion with wings hinged thereto; each wing has a plurality of legs connected to it by respective pin connections, so that the legs can be folded from a deployed configuration to a compact storage configuration.

This application claims benefit of provisional application 61/995110, filed Apr. 4, 2014.

### BACKGROUND OF THE INVENTION

This invention relates to an expandable and foldable mayo stand.

The nature of the mayo stand has been limited to its initial design in 1930. The basic design allows for ease of use in a tight space with instruments placed in reach of a surgeon on a movable stand.

The conventional mayo stand has a biped footplate attached to a vertical strut. At the top, a plate extends perpendicular to the strut in the same direction as the footplate. The strut can be lengthened, or lowered to accommodate patient and operating table position.

Instruments can be placed on top of the mayo tray but are limited to those without significant weight. Recent reinforced mayo stands have allowed for higher weight usage. Additional modifications have included double struts as well as a more robust footplate for a more rigid platform.

Conventional mayo stands cannot be reconfigured, and take up considerable valuable space when not in use.

### SUMMARY OF THE INVENTION

An object of the invention is to reduce the space needed for storage of a mayo stand.

A related object is to make a mayo stand easier to ship and move about.

A further object is to increase the utility of a mayo stand in an operating room.

These and other objects of the invention are attained by the invention described below.

These and other objects are attained by an expandable and foldable mayo stand as described below.

### BRIEF DESCRIPTION OF THE DRAWINGS

In the accompanying drawings,

FIG. 1 is a perspective view of an expandable and foldable mayo stand embodying the invention, with its legs and top in its open configuration;

FIG. 2 is a view like FIG. 1, showing the leg and top in a collapsed configuration;

FIG. 3 is a perspective view of an alternative embodiment of the invention in an open configuration; and

FIG. 4 is a view of the alternative embodiment in its collapsed configuration.

### DESCRIPTION OF THE PREFERRED EMBODIMENT

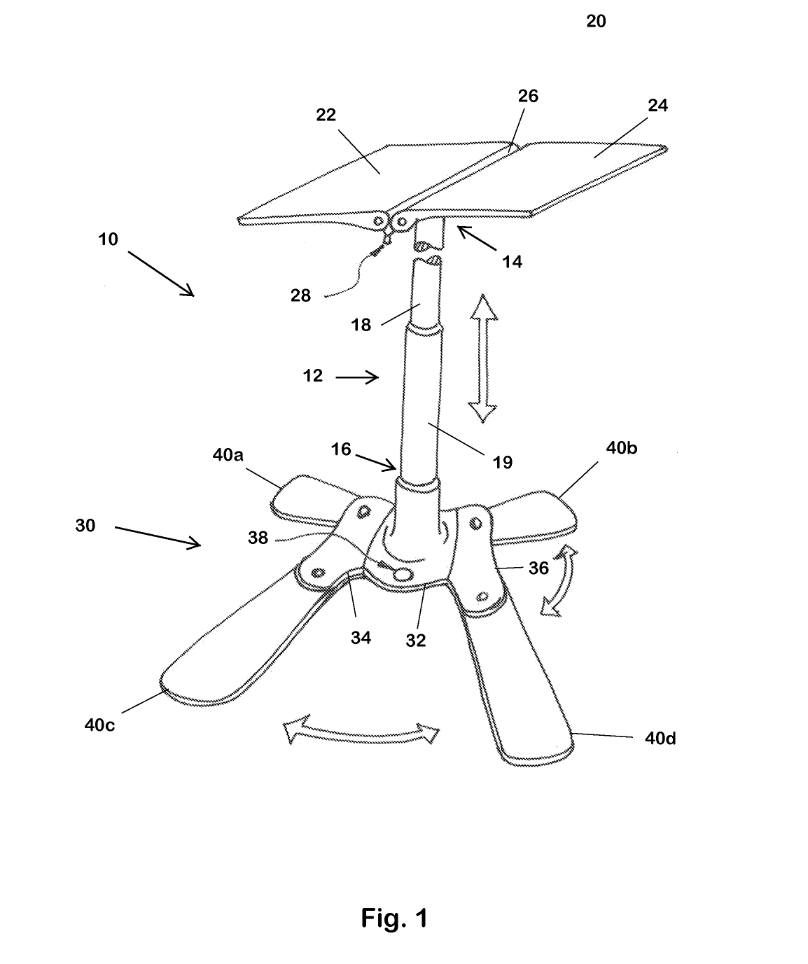

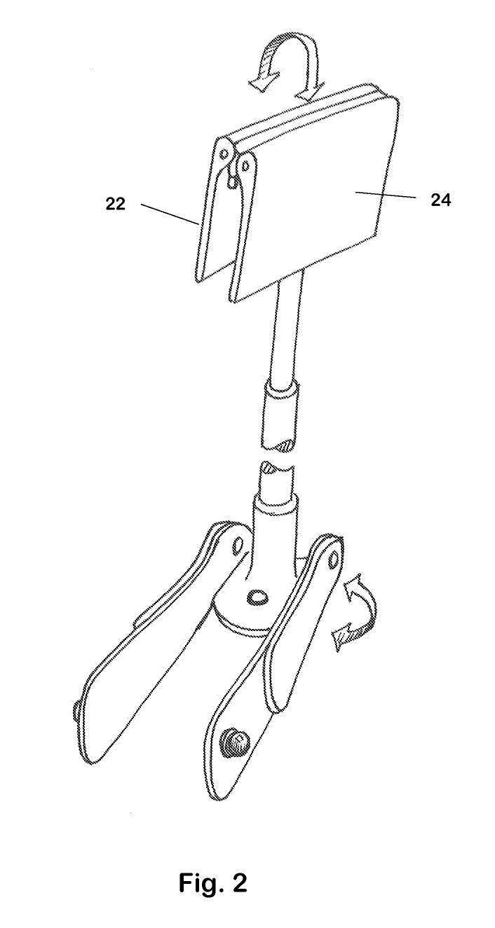

An expandable and foldable mayo stand embodying the invention is shown in FIGS. 1 and 2.

The mayo stand 10 includes a support column 12 having first (upper) and second (lower) ends 14 and 16 and a longitudinal axis “A”. The column is formed of at least two telescoping tubes 18, 19 which can move relatively so that the height of the stand can be adjusted.

A table top 20 is connected to the first end of the support column, and a base 30 is connected to the second end of the support column.

The table top 20 is formed in two leaves 22, 24 having a hinge connection 26 extending along a hinge axis “H” perpendicular to the longitudinal axis“A”. When a release lever 28 is operated, the leaves 22, 24 can be pivoted between a deployed position in which they are coplanar (FIG. 2) and a collapsed position (FIG. 2) in which they are substantially parallel and adjacent to the column 12. When the lever 28 is released, the leaves are held in their selected position.

The base 30 itself is foldable, having a central portion 32 and lateral wings 34, 36 which in their deployed positions are coplanar with the central portion, but can pivot upward about 90° when a release button 38 on the central portion is depressed.

Each of the base wings supports a pair of foldable legs. The legs are identified by reference numerals 40a, 40b, 40c and 40d. Each leg is connected to one of the base wings by a pin 42. A detent of locking mechanism (not shown) might be provided to hold the legs in their folded (FIG. 1) or deployed (FIG. 2) positions.

The table top of the mayo stand can be altered to create additional space in the OR (operating room) during storage of the Mayo stand. It also allows one to increase workable space during the OR case. Alterations in draping techniques and drape designs would complement the alterations to the mayo stand.

The mayo table leaves store in a vertical position. When placed into service in an operating room, the workable surface if lifted from the vertical position into a horizontal plane.

The table top can be outfitted with hook and capture arrays that would allow for additional positioning, stacking, and sorting of instruments.

One embodiment of the stand has articulating feet that would fold up to allow a reduced storage foot print while maintaining the highest degree of overall table top stability.

When in the usable position, separate feet would engage the floor in a XH configuration, with the lower arms longest under the operator to allow for greater stability of the entire mayo stand.

When in the storable position, the XH would fold to form two paralleling legs on edge in a horizontal plane, and finally pivot to settle in a vertical position running parallel with the main telescopic vertical column

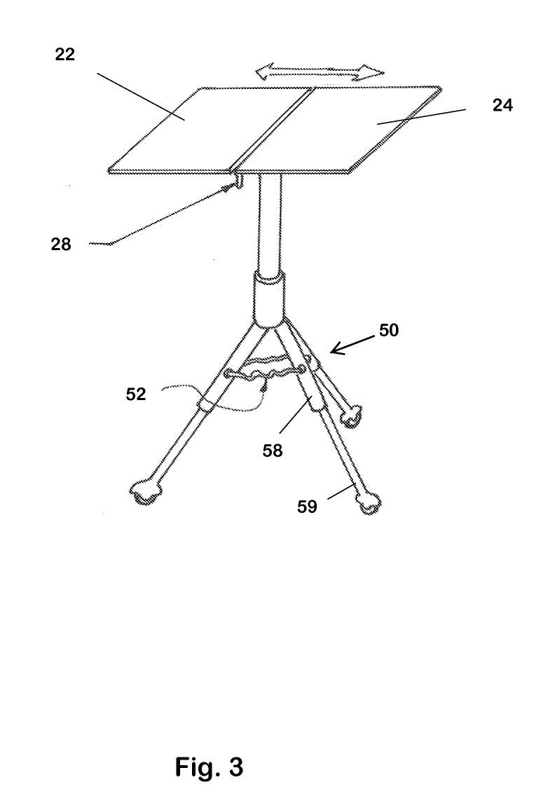

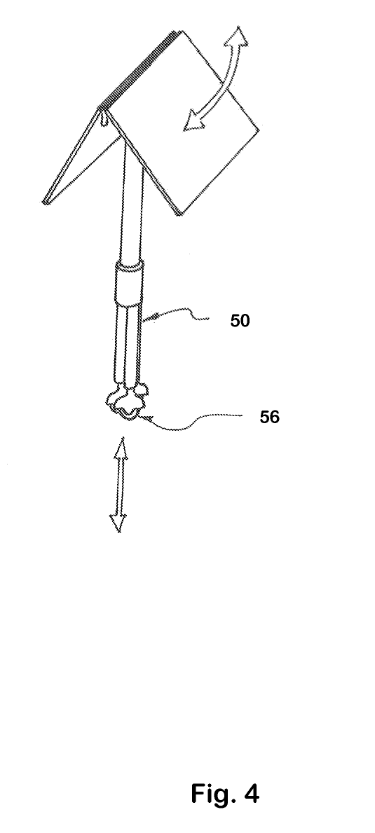

An alternative embodiment of the invention is shown in FIGS. 3 and 4. In this embodiment, there are a plurality (preferably three) legs 50 that store in the vertical central column via a telescopic mechanism. When deployed, the legs extend a distance limited by a restraint 52 such as a cable, and their distal feet 54 separate from each other forming a triangular or tripod-like base. Each foot may be provided with a caster 56 or other rolling mechanism.

Preferably, each of the legs itself has telescoping elements 58, 59 so that the leg length can be independently adjusted to alter the table top height, and to enable one to tilt the table top from horizontal.

Since the invention is subject to modifications and variations, it is intended that the foregoing description and the accompanying drawings shall be interpreted as only illustrative of the invention defined by the following claims.

I claim as my invention:

1. An expandable and foldable mayo stand comprising
a support column having first and second ends and a longitudinal axis,
a table top connected to the first end of the support column, and
a base connected to the second end of the support column, wherein
the table top is formed in two halves having a hinge connection extending along a hinge axis perpendicular to said longitudinal axis, whereby the table top halves can be pivoted between a deployed position in which they are coplanar and a collapsed position in which they are substantially parallel, and
the base has a plurality of legs connected thereto by respective pin connections, each of said legs being foldable about its pin connection between retracted and extended configurations.

2. The invention of claim 1, wherein the support column comprises plural telescoping tubes.

3. The invention of claim 1, wherein each of said legs comprises plural telescoping members.

4. The invention of claim 1, wherein the base has a central portion and a plural lateral wings which are hinged to the central portion so that they can fold upwardly from a deployed position out of the plane of the central portion to a storage position.

5. The invention of claim 1, further comprising a locking mechanism for holding the wings in their deployed position, said locking mechanism including a release button which can be depressed to unlock the wings.

6. The invention of claim 1, wherein each of the legs can be stored within the column.
